(19) United States
(12) Patent Application Publication
(10) Pub. No.: US 2005/0048136 A1
(43) Pub. Date: Mar. 3, 2005

(54) Rehydrating beverage with Rhodiola crenulata and D-ribose that enhances blood oxygen and relieves post-exertional muscle cramping and soreness

(76) Inventor: Muhammad S. Choudhry, Algonquin, IL (US)

Correspondence Address: MUHAMMAD S. CHOUDHRY, 1420 - Brandywine Cir, Algonquin, IL, 60102, US

(21) Appl. No.: 10/648,648

(22) Filed: Aug. 27, 2003

(51) Int. Cl. A61K 35/78; A61K 31/70

(52) U.S. Cl. 424/725; 514/23

(57) ABSTRACT

This invention relates to a rehydrating beverage containing Rhodiola crenulata and D-Ribose, which enhances blood oxygen, working capacity and endurance of exercising subjects. The rehydrating beverage containing Rhodiola crenulata and D-Ribose administered before a workout reduces muscle fatigue, prevent or alleviate muscle cramping and soreness in exercising subjects.

The rehydrating beverage was flavored, sweetened with sucralose with or without carbonation, contained specified amounts of herbal extract Rhodiola crenulata a simple sugars, D-ribose and glucose, electrolytes, and B-complex vitamins.

Rhodiola crenulata is an adaptogenic herb that offers varied physiological support to numerous systems in the body. D-ribose, or simply ribose, is a basic carbohydrate regulates the production of ATP, the most basic source of all cellular energy. The use of Rhodiola crenulata and D-ribose in re-hydrating beverage promotes blood oxygen levels, to enhance working capacity and endurance, to enhance cardiac and cardiovascular function, to prevent and alleviate of muscle cramping and soreness in exercising subjects.

### TECHNICAL FIELD

This invention relates to a re-hydrating beverage developed with Rhodiola crenulata and D-ribose, with or without caffeine, and electrolytes; to increase blood oxygen, to lessen muscle soreness and cramps and useful to enhance working capacity and overall physical energy level.

### BACKGROUND OF THE INVENTION

In reference to U.S. Pat. No. 4,853,237 & U.S. Pat. No. 4,874,606 the rehydrating drinks customarily comprising of carbohydrates (simple sugar or glucose polymer), electrolytes, such as sodium, potassium, and fruit acid. This invention approached in a unique way that will enhance the blood oxygen and to lessen muscle soreness and cramping to enhance working capacity and endurance of exercising subjects.

The present invention suggests to a combined usage of Rhodiola crenulata with D-ribose, with or without caffeine into a beverage for a variety of useful and beneficial effects, including oxygenation of blood through enhancing blood transport, to enhance working capacity and endurance, to reduce muscle fatigue and soreness, to increase alertness, to provide antioxidant effects, to protect against oxidation.

U.S. Pat. No. 6,399,116 to Xiu, discloses the use of Rhodiola crenulata to treat various conditions and diseases in mammals. Rhodiola crenulata is a Tibetan herb and is preferred to enhance blood oxygen levels, to enhance working capacity and endurance, to enhance memory and concentration, to enhance cardiac and cardiac function, to provide antioxidant effects, to protect against oxidation.

Yin, et al demonstrated that Rhodiola crenulata has the functions of adjusting central nervous systems, kinetic capacities, physical metabolism and anti-anoxia.

U.S. Pat. No. 6,159,942 to St. Cye discloses that precursors of adenosine triphosphate are administered orally to increase intracellular ATP concentration as dietary supplements or for treatment of reduced energy availability resulting from strenuous physical activity, illness or trauma. Pentose sugars are administered individually, mixed into dry food or in solution. The preferred pentose is D-ribose, singly or combined with creatine, pyruvate, L-carnitine and/or vasodilating agents. The compositions and methods of this invention are especially beneficial to mammals having reduced energy availability or high-energy demand.

As disclosed in the Patent that ribose has been found to be useful in enhancing energy in health male patients with cardiovascular disease, or peripheral vascular disease. It has likewise been discovered that the presence of ribose during a hypoxic event can raise the hypoxic threshold in persons encountering situations of low oxygen availability.

### SUMMARY OF THE INVENTION

The present invention relates to the beverage compositions, methods of use, methods of treatment, methods of preparation, etc. which relates to a combination of D-ribose and Rhodiola crenulata, which have a variety of useful and beneficial effects, including, e.g. to prevent and alleviate muscle cramps and soreness, to enhance blood oxygen and nutrients levels, e.g., through enhancing blood oxygen transport, to enhance working capacity and endurance, to enhance cardiac and cardiovascular function, to provide antioxidant effects, to protect against oxidation. The present invention provides combination of Rhodiola crenulata and D-ribose alone or in combination with other ingredients such as electrolytes, B-complex vitamins, and other carbohydrates, to prevent and alleviate muscle cramps and soreness, to enhance blood oxygen, and to provide overall physical energy.

It has been found that the addition of a specific amount of Rhodiola and D-ribose in combination with electrolytes, with or without caffeine, B-complex vitamins, glucose and a sweetener such as sucralose (a non nutritive sweetening agent) increases the blood oxygen and to prevent and alleviate the muscle soreness and cramping in exercising subjects. The addition of sucralose without adding the calories made the beverage more palatable and acceptable by the calorie conscious consumer.

The products with the scope of this invention may take a variety of forms. For instance, the product may be manufactured and sold as a single strength beverage as a ready to drink beverage by the consumer. Alternatively, the product may be in the from of elixir an aqueous syrup with higher concentration of nutrients with or without diluting the concentrate. The product may also come in dry powder or tablet form, which may be dissolved into a glass of water or taken with water to yield the maximum effect of this invention.

### DETAILED DESCRIPTION OF THE INVENTION

The present invention relates to beverage compositions, articles of manufacture, extracts, compounds, methods of use, methods of treatment, methods of preparation, etc., which relate to the combination Rhodiola crenulata and D-ribose, which have a variety of useful and beneficial effects. The combination of Rhodiola crenulata and D-ribose of the present invention can be used in any form which is effective, including, but not limited to liquid beverage RTD, dry powder, tablet, extracts, and other conventional compositions.

Rhodiola crenulata and D-ribose can be combined with electrolytes, caffeine, vitamins, and carbohydrates, such as glucose, sucralose and/or aspartame, non-nutritive sweeteners. A combination of Rhodiola crenulata and D-ribose can also be formulated with other active ingredients such as L-camitine, creatine monohydrate, pyruvate, alpha lipoic acid, Co Enzyme Q10, NAD, and NADH and antioxidants, such as reserveratrol and/or ginseng.

The present invention relates to administering Rhodiola crenulata and D-ribose, has been found e.g., to enhance blood oxygen levels, to be useful in enhancing energy in healthy males, to enhance working capacity and endurance, to reduce muscle fatigue, to reduce stress, to enhance cardiac and cardiovascular function, to improve sexual ability, and to provide antioxidant effects.

The present invention also discovered that the presence of Rhodiola crenulata and D-ribose during a hypoxic event could raise the hypoxic threshold in a person encountering situations of low oxygen availability. It has also been discovered that the combination of Rhodiola crenulata and D-ribose can also relieves exercise induced-muscle cramping and soreness. It is believed that the use of a beverage containing Rhodiola crenulata and D-ribose during exercise relieves the immediate effect of exercise, that is cramping and soreness, while replenishing nutrients of Rhodiola and D-ribose in the muscle by the administration of Rhodiola and D-ribose after exercise also relieves the later developing of soreness.

The rehydrating beverage containing Rhodiola crenulata and D-ribose promotes the prevention or alleviation of muscle cramping and soreness induced by exercise, alone or in combination with electrolytes, caffeine, vitamins and other carbohydrates. This invention also provides doses and protocols for maximum beneficial effect.

Administration of Rhodiola crenulata and D-ribose as a beverage over a period of 30 days can also be used to enhance blood oxygen levels, to enhance working capacity and endurance. By “working capacity and endurance,” it preferably meant that the effective amount increases the ability to perform a physical task, such as in exercise, physical labor, or sports activity, even before fatigue would normally occur; thus, enhanced working capacity and endurance is defined herein not to mean “fatigue,” which simply indicates that the performer does not tire. Associated with enhanced working capacity and endurance can be an increase in muscle ATP levels and a reduction in circulating lactic acid levels (see Examples 2 and 3). Thus, the present invention also relates to methods of increasing muscle ATP levels and/or reducing lactic acid in blood, thereby enhancing working capacity and endurance, etc,

### EXAMPLES

### Method of Examples One and Two

### Example One

Twelve exercising subjects are equally divided into four group's male and female varying from 23 to 45 years of age. Group A (male) and group B (female) were given a orange flavored low calorie beverage (sweetened with sucralose) containing Rhodiola crenulata and D-ribose and group C (male) and group D (female) were given orange flavored electrolyte drink similar in composition with Gatorade for a month. Using a blood oxygen analyzer, (Nonin Pulse Oximeter) the blood oxygen pressure of each participant before and after taking the beverage containing a combination of Rhodiola crenulata and D-ribose based beverage and placebo was tested. The average of the blood oxygen pressure of the test group before and after taking Rhodiola crenulata and D-ribose beverage for a month was 68.6.+−0.51 and 78.2+−.0.62 respectively. For the group, (taking placebo that is electrolyte beverage) the average level before and after taking placebo for a month was 67.85+−.0.49 and is 70.55+−.0.61 respectively. These results show after taking a beverage containing Rhodiola crenulata and D-ribose for one month, the oxygen transport and supply in the human body is enhanced by about 13.90%.

### Example Two

Test subjects that took beverage containing Rhodiola crenulata and D-ribose expressed that they felt improved sexual performance after one week drinking the beverage. The control group that took electrolyte beverage expressed no change in their sexual performance. The test group was asked if they felt physically strong after taking the beverage containing Rhodiola crenulata and D-ribose. The 68% of the test subjects reported improvement.

### Example Three

An experienced male athlete aged 52 that have been working out in the gym for last 9 months regularly. His training regimen consists of three days of strength training and three days of treadmill at a speed of 6.5 miles per hour. During the training, he has experienced varying degrees of muscle cramping and soreness. He was asked to take two bottles of beverage containing Rhodiola crenulata and D-ribose at a concentration of 0.50 grams of each ingredient 15-minute before training started. In addition, Rhodiola crenulata and D-ribose the beverage also contained electrolytes, B-complex vitamins, caffeine, and sucralose. He stated that after three days of taking the beverage, which contained Rhodiola crenulata and D-ribose he never noticed cramping, soreness of muscle and never felt fatigue.

### Example Four

Rhodiola crenulata and D-ribose containing rehydrating beverage reduces blood lactic acid (lactate) levels in exercising subjects. Such reduction in lactic acid levels indicates reduction in muscle cramping and soreness. A study was conducted with bicyclists that showed “lactic acid” was reduced 40% during strenuous exercise in athletes who took rehydrating beverage containing Rhodiola crenulata and D-Ribose.

Lactic acid is a natural compound produced in the “muscles” during exercise. When it accumulates to high levels it can be one of the causes of painful muscle cramps, spasms, sore muscles and can prolong recovery time between workouts. Lactic acid increase frequently force athletes to leave the field of play to sit out the game until the muscle cramps are relieved. It can also affect men and women in fitness training. The study was conducted with healthy male bicyclists who took rehydrating beverage before racing on stationary bicycles during a 30 K trial run compared to bicyclists who took placebos, (an electrolyte beverage). One week later the test group took placebos and the placebo group took the rehydrating beverage containing Rhodiola crenulata and D-Ribose. “In summary, rehydrating beverage positively affected the lactate metabolism during exercise and induced a glycogen-sparing effect and therefore benefited performance and endurance in exercising subjects.

The present invention therefore relates to a method of reducing muscle fatigue by administering an effective amount of Rhodiola crenulata in combination with D-ribose. Lactic acid can be reduced by an effective amount in curing fatigue, such as at least 25% to 40%, etc.

### Example Five

A rehydrating beverage comprising of effective amounts of Rhodiola crenulata and D-ribose can be administered to subjects to enhance levels of blood oxygen, to enhance working capacity and endurance, to reduce stress, to enhance cardiac and cardiovascular Function, to provide antioxidant effects, to increase energy level and to lessen from exercise-induced muscle soreness and cramping.

Without further elaboration, it is believed that one skilled in the art can use the preceding description, utilize the present invention to its fullest extent. While the compositions and methods of this invention have been described in terms of preferred embodiments, it will be apparent that variations may be applied to the compositions and methods described herein without departing the concept and scope of the invention.

1. A method of enhancing blood oxygen in exercising-induced subjects complaining muscle cramping and soreness by administering rehydrating beverage with an effective amount of Rhodiola crenulata and D-ribose a sufficient time prior to exercising whereby the effect of Rhodiola crenulata and D-ribose is effective to enhance blood oxygen and to reduce blood levels of Lactic acid.

2. The method according to claim 1 where the amount of Rhodiola crenulata is from 0.1 to 5 grams.

3. The method according to claim 1 where the amount of D-ribose is from 0.125 grams to 5 grams.

4. The method according to claim 1 where the amount of Rhodiola crenulata combined with D-ribose is used from 0.1 gram to 5 grams.

5. A flavored and sweetened aqueous rehydrating beverage comprising of an effective amount of Rhodiola crenulata and D-ribose.

6. The method according to claim 5 the amount of Rhodiola crenulata and D-ribose is from 0.1 to 5 grams each.

7. A rehydrating beverage according to claim 5 which further comprises about 1.00 to 2.5% glucose.

8. A rehydrating beverage according to claim 5, which further comprises sucralose as a sweetener about 0.01 to 0.02%.

9. A rehydrating beverage according to claim 5 which further comprises about 0.03 to 0.05% of caffeine.

10. A rehydrating beverage according claim 5 which further contains effective amount of sodium, citric acid, and flavoring agent.
